(19) United States
(12) Patent Application Publication
(10) Pub. No.: US 2012/0123239 A1
(43) Pub. Date: May 17, 2012

(54) Medical Image Processing System and Processing Method

(75) Inventors: Dae Hee Han, Seoul (KR); Jong Hyo Kim, Seoul (KR)

(73) Assignees: CAHOLIC UNIVERSITY INDUSTRY ACADEMIC COOPERATION FOUDATION, Seoul (KR); SNU R&DB FOUNDATION, Seoul (KR)

(21) Appl. No.: 13/319,303

(22) PCT Filed: May 7, 2010
(86) PCT No.: PCT/KR10/02906

§ 371 Date: Jan. 20, 2012

(30) Foreign Application Priority Data
May 8, 2009 (KR) 10-2009-0040218

(51) Int. Cl. A61B 6/00

(52) U.S. Cl. 600/407

(57) ABSTRACT

According to the invention, a medical image processing system comprises: a storage unit storing a medical image, a database server storing medical image information about the medical image, a computer aided diagnosis unit identifying an object to be identified and surrounding objects of the object using the medical image and the medical image information, and a position detector detecting a relative position of the identified object on the basis of the surrounding objects using the identified object and surrounding objects and storing information about the detected position. Accordingly, the medical image processing system can detect and provide the position of the object, and the medical image can be rapidly accurately interpreted at a hospital.

### CROSS-REFERENCE TO RELATED APPLICATION

This application claims priority to and the benefit of Korean Patent Application No. 2009-0040218, filed on May 8, 2009, the disclosure of which is incorporated herein by reference in its entirety.

### BACKGROUND 1. Field of the Invention

The disclosed technology relates to a medical image processing system and processing method, and more particularly, to a medical image processing system and processing method capable of reducing time taken by a radiologist to interpret a medical image and easily checking an interpreted result using only positional information even when there is no medical image.

2. Discussion of Related Art

As telecommunication technology is applied to various industrial fields, technology for storing and managing information is developing in the respective fields. For example, a digital picture archiving and communication system (PACS), by which medical images can be stored and managed at a hospital, has been introduced in the medical industrial field. The PACS converts medical images, which are acquired by capturing body regions of a patient using various types of medical equipment, into digital data, and stores the digital data in a storage medium.

Medical doctors can refer to and check desired medical images, history, etc. of a patient via a computer monitor in hospital clinics. Further, medical radiologists can interpret the current state or disease of a patient using medical images, and carry out measures required for care or treatment of the patient according to the interpreted result.

### SUMMARY OF THE INVENTION

The disclosed technology is directed to a medical image processing system and processing method capable of reducing time taken by a radiologist to interpret a medical image and the burden on business of the radiologist.

The disclosed technology is also directed to a medical image processing system and processing method capable of easily checking an interpreted result using only position information even when the medical image is not interpreted due to a different data format thereof, or when there are no medical images.

The disclosed technology is also directed to a medical image processing system and processing method capable of providing information about from which region an object is frequently generated according to a type of the object.

According to an aspect of the disclosed technology, there is provided a medical image processing system, which comprises: a storage unit storing a medical image of lungs of a patient; a database server storing medical image information about the medical image; a computer aided diagnosis unit identifying a pulmonary vein and a pulmonary nodule from the medical image using the medical image and the medical image information; and a position detector detecting a relative position of the pulmonary nodule on the basis of the pulmonary vein using the identified pulmonary nodule and vein, and storing information about the detected position.

According to another aspect of the disclosed technology, there is provided a medical image processing system, which comprises: a storage unit storing a medical image of lungs of a patient; a database server storing medical image information about the medical image; a clinical interpretation station which displays the medical image and the medical image information on a screen and at which a nodule whose position is to be detected from the medical image is selected; and a position detector identifying the nodule whose position is to be detected and a pulmonary vein using the medical image and the medical image information, detecting a relative position of the identified nodule on the basis of the pulmonary vein using the identified nodule and pulmonary vein, and storing information about the detected position.

According to yet another aspect of the disclosed technology, there is provided a medical image processing system, which comprises: a storage unit storing a medical image; a database server storing medical image information about the medical image; a computer aided diagnosis unit identifying an object to be identified and surrounding objects of the object using the medical image and the medical image information; and a position detector detecting a relative position of the identified object on the basis of the surrounding objects using the identified object and the identified surrounding objects and storing information about the detected position.

According to still yet another aspect of the disclosed technology, there is provided a medical image processing system, which comprises: a storage unit storing a medical image; a database server storing medical image information about the medical image; a clinical interpretation station which displays the medical image and the medical image information on a screen and at which an object whose position is to be detected from the medical image is selected; and a position detector identifying the object whose position is to be detected and surrounding objects using the medical image and the medical image information, detecting a relative position of the identified object on the basis of the surrounding objects using the identified object and surrounding objects, and storing information about the detected position.

According to still yet another aspect of the disclosed technology, there is provided a medical image processing method, which comprises: acquiring and storing a medical image; identifying an object to be identified and surrounding objects of the object using the medical image and the medical image information; detecting a relative position of the identified object on the basis of the surrounding objects using the identified object and surrounding objects; and storing information about the detected position.

According to the disclosed technology, since the medical image processing system detects and provides the relative position using the surrounding objects, a radiologist can accurately rapidly interpret the medical images even when the medical image is captured several times or using a different imaging instrument or hospital. Thus, the radiologist can reduce time required to interpret the medical images, and the burden on business.

Since the medical image processing system according to the disclosed technology stores the labeling information as the position information, it is possible to easily check an interpreted result using only position information even when the medical image is not interpreted due to a different data format thereof, or when there are no medical images.

Further, the medical image processing system according to the disclosed technology can database object-specific position information to provide information about position tendency of the object. Thus, the medical image processing system can provide information about from which region the object is frequently generated according to a type of the object using the position tendency information.

### BRIEF DESCRIPTION OF THE DRAWINGS

The above and other objects, features and advantages of the disclosed technology will become more apparent to those of ordinary skill in the art by describing in detail exemplary embodiments thereof with reference to the accompanying drawings, in which:

FIGS. 1 and 2 show an example of a medical image;

FIG. 3 is a view for explaining a medical image processing system according to an exemplary embodiment of the disclosed technology;

FIG. 4 is a view for explaining a medical image display screen according to an embodiment of the disclosed technology;

FIG. 5 shows an example of a medical image according to an embodiment of the disclosed technology;

FIG. 6 shows an example of a position detecting method according to an embodiment of the disclosed technology; and

FIG. 7 is a flowchart showing a process of processing a medical image using a medical image processing system according to an embodiment of the disclosed technology.

### DETAILED DESCRIPTION OF EXEMPLARY EMBODIMENTS

Exemplary embodiments of the disclosed technology will be described in detail below with reference to the accompanying drawings. However, specific structural and functional details disclosed herein are merely representative for purposes of describing example embodiments. The disclosed technology, however, should not be construed as limited to only example embodiments set forth herein. Accordingly, it should be understood that, since example embodiments are capable of various modifications and alternative forms, they are to cover all modifications, equivalents, and alternatives falling within the scope of the disclosed technology.

Unless otherwise specified in the context, the steps may be performed out of the specified order. Accordingly, the steps may be performed in the same order, be performed substantially concurrently, or be performed in the reverse order.

Unless otherwise defined, all terms used herein, including technical or scientific terms, have the same meanings as those generally understood by those with ordinary knowledge in the field of art to which the disclosed technology belongs. Such terms as those defined in a generally used dictionary are to be interpreted to have the meanings equal to the contextual meanings in the relevant field of art, and are not to be interpreted to have ideal or excessively formal meanings unless clearly defined in the present application.

Various medical images of a patient are captured and stored at a hospital, so that a state of the patient can be determined by the medical images. Instruments capable of capturing such medical images include, for example, various radiological imaging instruments such as a computed tomography (CT) instrument, a magnetic resonance imaging (MRI) instrument, an X-ray instrument, an ultrasonography instrument, an angiography instrument, a colposcopy instrument, a cervicography instrument, and so on, in addition to nuclear medicine imaging instruments. The captured medical images are converted into digital data, and the digital data is stored and provided to hospital members via a picture archiving and communication system (PACS).

A radiologist who interprets medical images can refer to, check and interpret the medical images on a computer monitor via the PACS. The radiologist can access the PACS via a clinical interpretation station, and refer to and interpret medical images of a patient.

When a medical image is interpreted, the interpreted results may differ depending on capability or experience of the radiologist. Thus, even when a result has already been interpreted by a radiologist, a new radiologist must interpret the medical image again. Further, the medical image of a patient may be captured several times depending on the progress of a disease or using different equipment or a different hospital. Thus, various medical image data may be created, and the same affected region may appear to be in a different in position in the medical images. As such, whether or not the affected region is the same region must be interpreted each time.

In such a case, it may take the radiologist much time to interpret the medical image. It may be a burdensome job for the radiologist to interpret each medical image. For example, it is assumed that a radiologist interprets medical images of a patient having a nodule at a lower end of a left lung. When the medical image for the patient is captured at regular intervals, a plurality of medical images may be obtained.

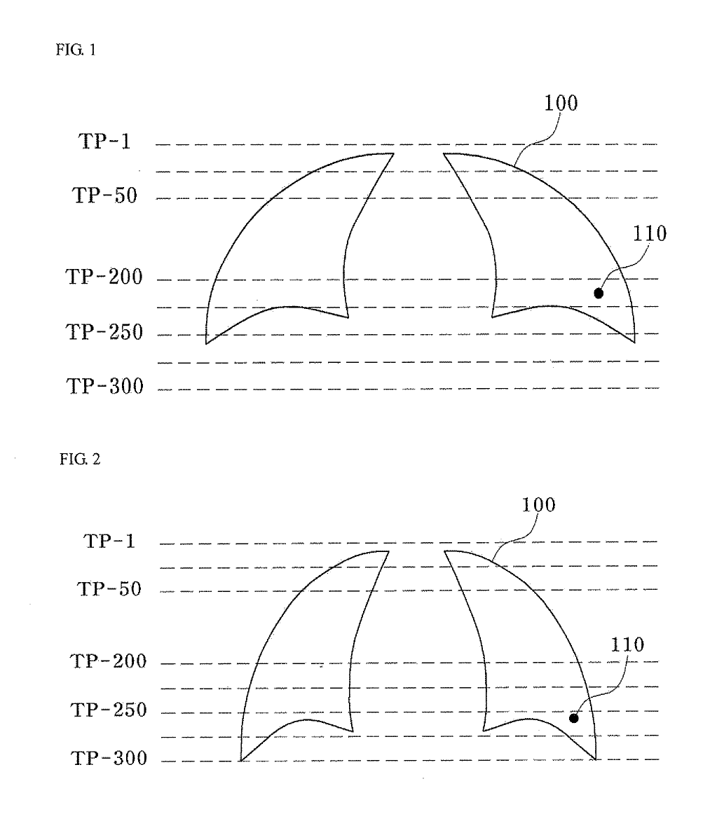

FIGS. 1 and 2 show an example of a medical image. It is assumed that the medical image of FIGS. 1 and 2 is a chest medical image captured from the lungs of a patient who has a nodule 110 at a lower end of a left lung 100. Here, a position, size, table position (TP) line, etc. of the lung are illustrated to explain medical imaging technology.

The image of FIG. 1 is captured when a patient has inspired, and can be interpreted to show that the nodule 110 is located directly below a line of TP-200. In contrast, the image of FIG. 2 is captured when a patient has expired, and can be interpreted to show that the nodule 110 is located below a line of TP-250.

Accordingly, when interpreting the image on the basis of only the TP line, a radiologist has to determine whether the nodule of FIG. 1 is the same as that of FIG. 2, and a long time is required for the interpretation. Further, when the interpretation is done by another radiologist, the result may differ. Furthermore, when the number of nodules is numerous, the interpretation may become more difficult.

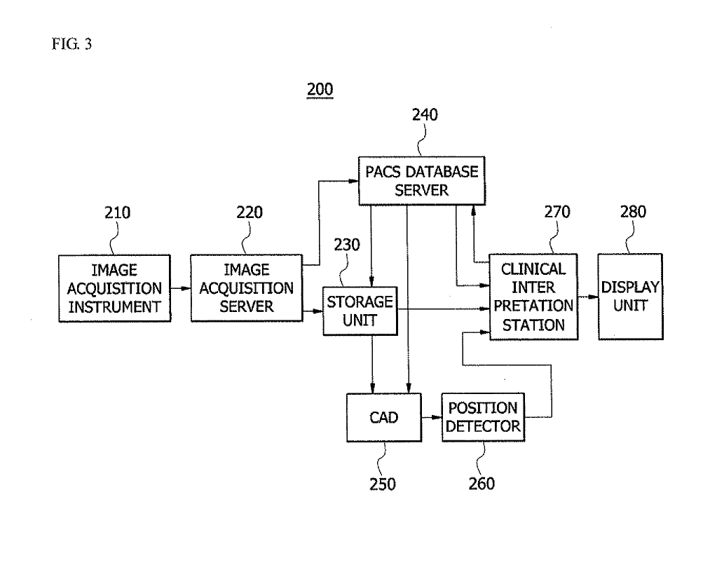

FIG. 3 shows configuration of a medical image processing system according to an exemplary embodiment of the disclosed technology. Referring to FIG. 3, the medical image processing system 200 includes an image acquisition instrument 210, an image acquisition server 220, a storage unit 230, a PACS database server 240, a computer-aided diagnosis (CAD) unit 250, a position detector 260, a clinical interpretation station 270, and a display unit 280. The medical image processing system 200 may further include an output unit (not shown) capable of outputting stored medical images, such as an optical disk output unit (not shown) or a memory output unit (not shown).

The image acquisition instrument 210 acquires medical images from patients. Examples of the image acquisition instrument 210 include various radiological imaging instruments such as a CT instrument, an MRI instrument, an X-ray instrument, an ultrasonography instrument, an angiography instrument, a colposcopy instrument and a cervicography instrument, and nuclear medicine imaging instruments.

When medical images captured in advance are received from a system outside the medical image processing system 200 or an external hospital and stored, the image acquisition instrument 210 may be a storage medium input unit such as an optical disk input unit or a memory input unit, or an image input unit such as a scanner.

The medical images acquired by the image acquisition instrument 210 are converted into digital data and stored. The image acquisition server 220 receives the medical images from the image acquisition instrument 210 and converts the received medical images into digital data.

The image acquisition server 220 may convert the medical images into the digital data according to a digital imaging communication in medicine (DICOM) format. DICOM refers to a standardized application layer protocol for transceiving medical images, waveforms, and incidental information. Alternatively, the image acquisition server 220 may use a separate format without using the DICOM format.

The image acquisition server 220 transmits the digitalized medical image and original image data to the storage unit 230. The image acquisition server 220 transmits medical image information about the medical images, such as storage path information of the image data, DICOM information, etc. to the PACS database server 240.

The storage unit 230 stores the digitalized medical image and original image data, and transmits the data by request.

The PACS database server 240 may store the medical image information such as storage path information of the image data, DICOM information, etc. of the image data received from the image acquisition server 220. Further, the PACS database server 240 may store image interpretation information, accessory mark information about the image on which a lesion is marked, identification information for identifying a patient, etc., all of which are received from the clinical interpretation station 270.

The clinical interpretation station 270 can provide access to the PACS database server 240, and refer to the medical images. A radiologist can refer to and interpret medical images of a patient using the clinical interpretation station 270. For example, a radiologist can refer to and interpret medical images of a patient using identification information (identifier (ID), resident number, name, birthdate, etc.) of the patient. Further, the clinical interpretation station 270 can store image interpretation information interpreted by the radiologist, accessory mark information about the image, etc. in the PACS database server 240.

When a radiologist makes a request for medical images of a patient, the clinical interpretation station 270 requests the storage unit 230 to transmit the corresponding medical images. The storage unit 230 transmits the requested medical images to the clinical interpretation station 270. The clinical interpretation station 270 displays information about the medical images received from the storage unit 230 and the medical images received from the PACS database server 240.

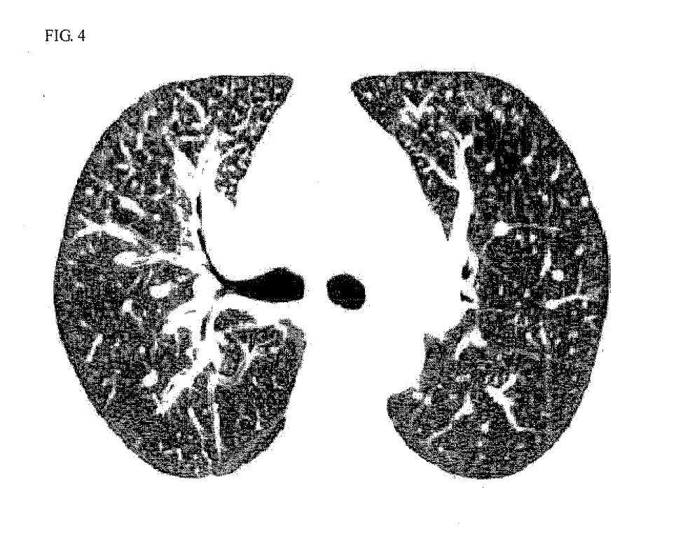

FIG. 4 is a view for explaining a medical image display screen according to an embodiment of the disclosed technology. Referring to FIG. 4, the medical image, information of a patient, information about a disease, etc. can be displayed on the display unit 280 as shown in FIG. 4. The screen of FIG. 4 is an example, and types of the information displayed on the display unit 280 may vary depending on a display mode. Further, the medical image processed in a different format, such as a two-dimensional image, a three-dimensional image, a specified organ extraction image, etc. may be displayed on the display unit 280 depending on the display mode.

The CAD unit 250 diagnoses medical images to provide diagnostic information. A radiologist can interpret the medical images with reference to the diagnostic information provided from the CAD unit 250. The radiologist may load only the medical images stored in the storage unit 230 onto the clinical interpretation station 270 to directly interpret the medical images, or drive a CAD function to interpret the medical images with reference to the diagnostic information.

The CAD unit 250 may identify a specified object or state of each medical image using anatomical information, and diagnose the medical image. The CAD unit 250 may select a diagnosis algorithm depending on a type of each medical image, or a feature of each object to be identified. For example, when the CAD unit 250 identifies and diagnoses a mass or nodule of a specified organ, the diagnosis algorithm may be selected depending on information about the specified organ, information about the mass or nodule of the specified organ, a type of the medical image, and so on. The diagnosis algorithm may be used to diagnose each medical image using various pieces of image information such as edge information, color information, strength change information, spectrum change information, image feature information, etc. of the medical image.

Although the objects of various organs can be interpreted using the anatomical information and the medical images, the following description is made for the sake of convenience under the assumption that a radiologist interprets pulmonary nodules from medical images of a patient. The radiologist may display and interpret only the medical images on the display unit 280, or drive a CAD function to interpret the medical images with reference to diagnostic information.

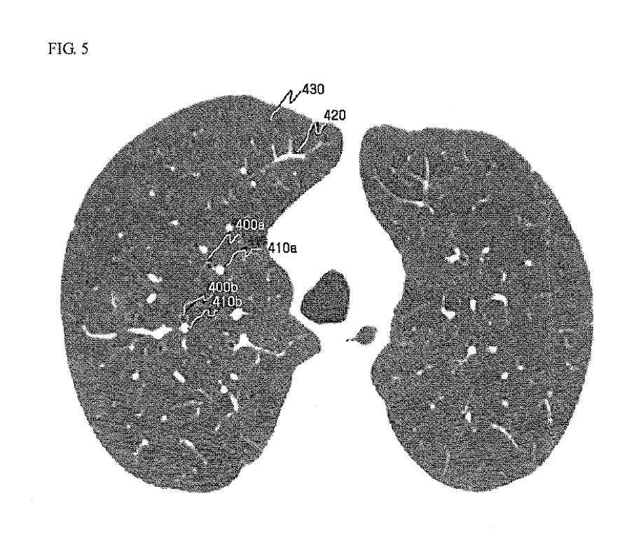

FIG. 5 shows an example of a medical image according to an embodiment of the disclosed technology. FIG. 5 shows one slice of a pulmonary image captured by a CT instrument. When a radiologist drives a CAD function, the CAD unit 250 identifies a specified object or state of each medical image using anatomical information, and diagnoses the medical image.

For example, the CAD unit 250 may identify and diagnose bronchi, pulmonary arteries, pulmonary veins, and nodules using the diagnosis algorithm. The bronchi 400a and 400b and the pulmonary arteries 410a and 410b are distributed through the lungs in pairs in close proximity to each other, and the pulmonary vein 420 is distributed through the lungs apart from the bronchi 400a and 400b or the pulmonary arteries 410a and 410b. Further, a pulmonary space 430 or the bronchi 400a and 400b which are filled with air may be shown in a color different from that of the pulmonary arteries 410a and 410b or the pulmonary vein 420 through which blood flows. The CAD unit 250 may identify the bronchi 400a and 400b, the pulmonary arteries 410a and 410b and the pulmonary vein 420 using the anatomical information and the image information as mentioned above.

Further, the CAD unit 250 may identify abnormal nodules in an anatomical aspect. For example, when objects to be identified are continuously connected to a plurality of image slices, the CAD unit 250 may identify them as the bronchi or blood vessels. Further, when objects to be identified are discovered from only image slices whose number is less than a predetermined number, the CAD unit 250 may identify the objects to be identified as the nodules. The CAD unit 250 may simultaneously identify a plurality of nodules.

The foregoing diagnosis algorithm is an example, and the nodules may be identified using other anatomical information or image information such as edge information, color information, strength change information, spectrum change information, image feature information, etc. of the medical image.

The CAD unit 250 may identify the bronchi, the blood vessels, the nodules, etc. from the medical images using the aforementioned method. The description is an example. When medical images of another organ are interpreted, the objects of the corresponding organ which are to be identified, such as nodules, may be identified.

The position detector 260 may identify positions of the objects using information about the objects identified by the CAD unit 250, and store the position information about the objects. The position detector 260 detects a relative position of each object to be detected using its surrounding objects. For example, the position detector 260 may detect the position information about the object on the basis of blood vessels, organs, and/or bones.

The following description is made under the assumption that the position detector 260 detects positions of pulmonary nodules from the pulmonary medical image as shown in FIG. 5 using relative positions of the pulmonary nodules to the pulmonary veins. The position detector 260 may detect the positions of. the nodules using various objects such as bronchi, pulmonary arteries, pulmonary veins, etc. identified from the lung. When the pulmonary veins and nodules are identified, the position detector 260 detects the relative position information about the nodules using one or more pulmonary veins.

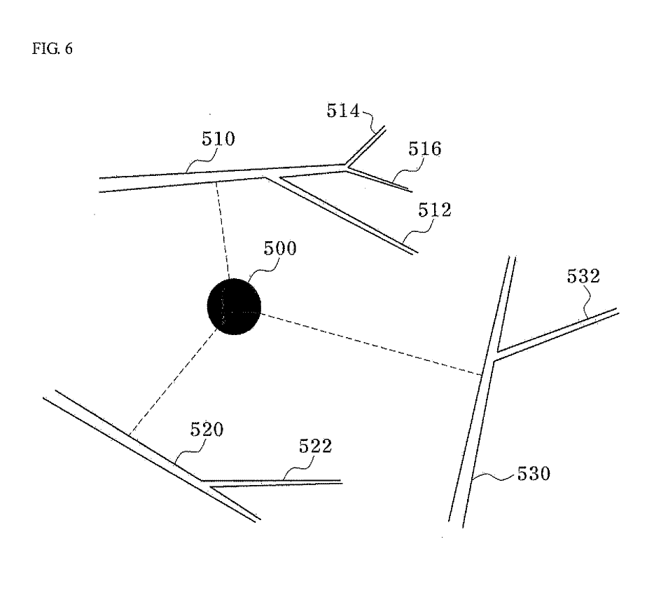

FIG. 6 shows an example of a position detecting method according to an embodiment of the disclosed technology. Referring to FIG. 6, a pulmonary nodule 500 is surrounded by three pulmonary veins 510, 520 and 530 in the medical image. The first pulmonary vein 510 has a first branch 512, a second branch 514, and a third branch 516. The second pulmonary vein 520 has a fourth branch 522, and the third pulmonary vein 530 has a fifth branch 532.

The position detector 260 detects a position on the basis of the pulmonary vein nearest the pulmonary nodule 500. The position detector 260 may detect a position on the basis of at least one pulmonary vein. The position detector 260 measures orthogonal distances between the pulmonary nodule and the pulmonary veins, and identifies at least one pulmonary vein having the shortest orthogonal distance. The position detector 260 may detect the pulmonary vein nearest the pulmonary nodule within the same image slice, or within several image slices in front and behind an image slice from which the pulmonary nodule is identified.

Referring to FIG. 6, the pulmonary nodule 500 is nearest the first pulmonary vein 510, the second pulmonary vein 520, and the third pulmonary vein 530 of the pulmonary veins. The position detector 260 stores information about the pulmonary veins nearest the pulmonary nodule 500.

The position detector 260 may label identification information for surrounding objects of the object whose position is to be detected, and store the labeled identification information. For example, the position detector 260 may label the identification information for the surrounding objects, i.e. the branches of each pulmonary vein, in order to detect the position of the pulmonary nodule 500 as shown in FIG. 6, and store the labeled identification information. A method of labeling the identification information may vary depending on an embodiment. However, the labeling is possible on the basis of anatomical classification. For example, when the position detector 260 labels the pulmonary veins as shown in FIG. 5, each pulmonary vein branch is labeled on the basis of the superior and inferior pulmonary veins of each of the left and right lungs.

When the first pulmonary vein 510 of FIG. 6 is made up of a first branch from the front of the inferior pulmonary vein of the left lung, a third branch among branches extending superiorly from the first branch, and a second branch among branches extending inferiorly from the third branch, the position detector 260 may label the first pulmonary vein 510 as “LIF1S3I2.” According to this labeling method, the third branch 516 of the first pulmonary vein 510 is the second one 516 of the branches 512 and 516 extending inferiorly from the first pulmonary vein 510, it can be labeled as “LIF1S3I2I2.”

The position detector 260 can label each pulmonary vein in the aforementioned method. When the first, second and third pulmonary veins 510, 520 and 530 are labeled as “LIF1S3I2,” “LIF2S3I1,” and “LIF3S1I2” in the aforementioned method respectively, the position detector 260 stores the labeling information about the first, second and third pulmonary veins 510, 520 and 530 nearest the pulmonary nodule 500 whose position is to be detected as the position information about the pulmonary nodule 500. A radiologist can use the position information to find that the pulmonary nodule 500 is located in a space surrounded by the first, second and third pulmonary veins 510, 520 and 530.

When the aforementioned labeling information is stored as the position information, it is possible to easily check the interpreted result using only the position information even when the medical images are not interpreted due to a different data format thereof, or there are no medical images.

In the example above, the position detector 260 obtains the position information using the three surrounding objects. However, the position information may be obtained using two or four or more surrounding objects depending on an embodiment. The position detector 260 may further include information about direction or distance of the object to be identified on the basis of the surrounding objects along with the position information as mentioned above.

The position detector 260 transmits the position information of the object to the clinical interpretation station 270, and the clinical interpretation station 270 can display the position information of the object on the display unit 280. A radiologist can check the position information of the object, and store the position information along with the image interpretation information and the accessory mark information in the PACS database server 240. The position detector 260 may directly store the position information of the object in the PACS database server 240. The PACS database server 240 may store types of the objects according to various medical image cases along with the position information of the objects as a database. The PACS database server 240 may provide information about position tendency of the object depending on a type of the object using the stored information. The PACS database server 240 provides the position tendency information, so that it can provide information about from which region an object is frequently generated according to a type of the object. For example, when the position of the pulmonary nodule is designated on the basis of a peripheral pulmonary vein, it can be more accurately checked which region of the lung is easily affected with a corresponding disease.

In the example above, the object is identified using the CAD unit 250, and then the position information of the object is detected and stored by the position detector 260. Alternatively, the object may be identified by a radiologist interpreting medical images, and then the position information of the object may be detected and stored by the position detector 260. That is, when a radiologist interprets medical images using the clinical interpretation station 270 and selects an object of interest as an object whose position is to be detected from the medical images, the position detector 260 may receive the medical images and information about the medical image from the storage unit 230 and the PACS database server 240, identify the object selected by the radiologist from the medical images, and detect position information of the object.

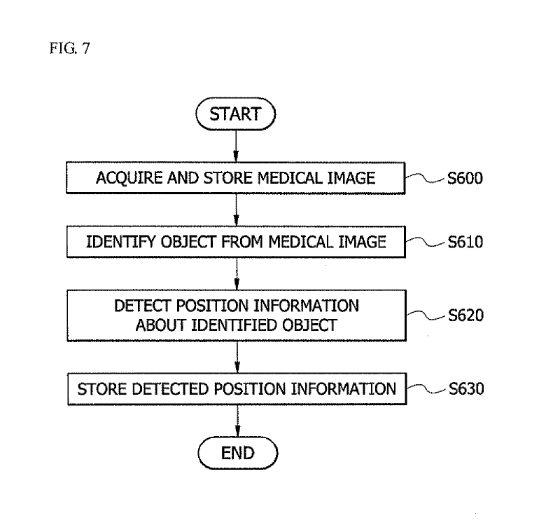

FIG. 7 is a flowchart showing a process of processing a medical image using a medical image processing system according to an embodiment of the disclosed technology. Referring to FIG. 7, the medical image processing system acquires and stores a medical image using an image acquisition instrument (S600). When a radiologist drives a CAD function, the CAD unit identifies an object using the medical image. A CAD unit may identify an object set to be identified by a radiologist using anatomical information and the medical image.

When the object is identified by the CAD unit, a position detector detects position information about the identified object (S620). The position detector can detect the position information using a position relative to surrounding objects, as described above. When the radiologist directly selects an object of interest from the medical image, the position detector may identify the object directly selected by the radiologist, and detect the position information of the object.

The position information detected by the position detector is stored in a PACS database server of the medical image processing system (S630). The PACS database server databases the position information, so that it can provide information about position tendency depending on the object. The stored position information may be provided to members within a hospital via a PACS of the medical image processing system, or be provided to other systems outside the medical image processing system via a storage medium such as an optical disk or a memory along with the medical image.

The medical image processing system according to an embodiment can detect and provide the positions of the objects. The medical image processing system according to the embodiment can automatically detect the positions of the objects.

Since the medical image processing system according to the embodiment detects and provides the relative position using the surrounding objects, a radiologist can rapidly interpret the medical images even when the medical image is captured several times or using a different imaging instrument or hospital. Thus, the radiologist can reduce time required to interpret the medical images, and the burden on business.

Since the medical image processing system according to the embodiment stores the labeling information as the position information, it is possible to easily check the interpreted result using only the position information even when the medical images are not interpreted due to a different data format thereof, or there are no medical images.

The medical image processing system according to the embodiment can database the object-specific position information to provide the position tendency information of the object. Thus, the medical image processing system can provide information about from which region the object is frequently generated according to a type of the object using the position tendency information.

It will be apparent to those skilled in the art that various modifications can be made to the above-described exemplary embodiments of the disclosed technology without departing from the scope of the disclosed technology. Thus, it is intended that the disclosed technology covers all such modifications provided they come within the scope of the appended claims and their equivalents.

What is claimed is:

1. A medical image processing system comprising:
a storage unit storing a medical image of lungs of a patient;
a database server storing medical image information about the medical image;
a computer aided diagnosis unit identifying a pulmonary vein and a nodule from the medical image using the medical image and the medical image information; and
a position detector detecting a relative position of the pulmonary nodule on the basis of the pulmonary vein using the identified pulmonary nodule and vein and storing information about the detected position.

2. The medical image processing system of claim 1, wherein the computer aided diagnosis unit identifies the pulmonary vein and nodule using anatomical information and the medical image information.

3. The medical image processing system of claim 1, wherein the position detector detects the relative position of the pulmonary nodule on the basis of at least one pulmonary vein near the pulmonary nodule.

4. The medical image processing system of claim 1, wherein the position detector labels identification information for each branch of the pulmonary vein, and stores the identification information of the at least one pulmonary vein near the pulmonary nodule as the position information of the pulmonary nodule.

5. The medical image processing system of claim 1, further comprising a clinical interpretation station that receives the position information from the position detector and displays the position information on a screen.

6. The medical image processing system of claim 1, wherein the database server receives and stores the position information and provides information about position tendency of the pulmonary nodule using the position information.

7. A medical image processing system comprising:
a storage unit storing a medical image of lungs of a patient;
a database server storing medical image information about the medical image;
a clinical interpretation station which displays the medical image and the medical image information on a screen and at which a nodule whose position is to be detected from the medical image is selected; and
a position detector identifying the nodule whose position is to be detected and a pulmonary vein using the medical image and the medical image information, detecting a relative position of the identified nodule on the basis of the pulmonary vein using the identified nodule and pulmonary vein, and storing information about the detected position.

8. The medical image processing system of claim 7, wherein the position detector detects the relative position of the nodule on the basis of at least one pulmonary vein near the nodule.

9. The medical image processing system of claim 7, wherein the position detector labels identification information for each branch of the pulmonary vein, and stores the identification information of the at least one pulmonary vein near the pulmonary nodule as the position information of the nodule.

10. The medical image processing system of claim 7, wherein the database server receives and stores the position information and provides information about position tendency of the nodule using the position information.

11. A medical image processing system comprising:
a storage unit storing a medical image;
a database server storing medical image information about the medical image;
a computer aided diagnosis unit identifying an object to be identified and surrounding objects of the object using the medical image and the medical image information; and
a position detector detecting a relative position of the identified object on the basis of the surrounding objects using the identified object and the identified surrounding objects and storing information about the detected position.

12. The medical image processing system of claim 11, wherein the position detector detects the relative position of the identified object on the basis of at least one surrounding object near the identified object.

13. The medical image processing system of claim 11, wherein the position detector labels identification information for each surrounding object, and stores the identification information of the at least one surrounding object near the identified object as the position information of the identified object.

14. The medical image processing system of claim 11, wherein the database server receives and stores the position information, databases the position information, and provides information about position tendency depending on the identified object.

15. A medical image processing system comprising:
a storage unit storing a medical image;
a database server storing medical image information about the medical image;
a clinical interpretation station which displays the medical image and the medical image information on a screen and at which an object whose position is to be detected from the medical image is selected; and
a position detector identifying the object whose position is to be detected and surrounding objects using the medical image and the medical image information, detecting a relative position of the identified object on the basis of the surrounding objects using the identified object and surrounding objects, and storing information about the detected position.

16. A medical image processing system that stores a medical image of an organ of a patient and information about the medical image, the medical image processing system comprising:
a position detector identifying a nodule and blood vessels of the organ, both of which are to be identified, using the medical image and the medical image information, detecting a relative position of the identified nodule on the basis of the blood vessels using the identified nodule and organ, and storing information about the detected position.

17. A medical image processing system that stores a medical image and medical image information about the medical image, the medical image processing system comprising:
a position detector identifying an object to be identified and surrounding objects using the medical image and the medical image information, detecting a relative position of the identified object on the basis of the surrounding objects using the identified object and surrounding objects, and storing information about the detected position.

18. A medical image processing method comprising:
acquiring and storing a medical image;
identifying an object to be identified and surrounding objects of the object using the medical image and the medical image information;
detecting a relative position of the identified object on the basis of the surrounding objects using the identified object and surrounding objects; and
storing information about the detected position.

19. The medical image processing method of claim 18, wherein the detecting of the relative position includes detecting the relative position of the identified object on the basis of at least one surrounding object near the identified object.

20. The medical image processing method of claim 18, further comprising databasing the position information and providing information about position tendency depending on the identified object.
